(19) United States
(12) Patent Application Publication
(10) Pub. No.: US 2008/0194517 A1
(43) Pub. Date: Aug. 14, 2008

(54) EQUINE OR CANINE IMMUNOMODULATING COMPOSITION AND TREATMENT METHOD

(75) Inventor: Larry G. Smith, Washington, PA (US)

Correspondence Address: BLYNN L. SHIDELER;THE BLK LAW GROUP, 3500 BROKKTREE ROAD, SUITE 200, WEXFORD, PA, 15090, US

(73) Assignee: L.C.M. Equine Nutraceuticals, Inc., Washington, PA (US)

(21) Appl. No.: 12/029,192

(22) Filed: Feb. 11, 2008

Related U.S. Application Data
(60) Provisional application No. 60/889,059, filed on Feb. 9, 2007.

(51) Int. Cl. A61K 31/716; A61P 37/02; A61P 31/00

(52) U.S. Cl. 514/54

(57) ABSTRACT

An equine or canine immunomodulating composition comprises a pre-packaged, feed supplement comprising effective amounts of a beta-glucan mixture which is at least 60 percent purified. The feed supplement may be a dry feed supplement further including (b) an effective amount of at least one flavoring, and (c) an effective amount of a sweetener. The feed supplement may include an effective amount of an antioxidant; and an effective amount of an emulsifier. An alternative composition further comprises an appetite stimulant. A method of treating equine or canine microbial infections following the diagnosis of a microbial infection comprises the step of treating the equine or canine for a period at least long enough to prevent immediate recurrence of the microbial infection with effective amounts of the immunomodulating composition described above. The immunomodulating composition may alternatively be used in combination with an effective amount of an antibiotic.

### RELATED APPLICATION

This application claims the benefit of provisional patent application Ser. No. 60/889,059 filed on Feb. 9, 2007 entitled “Equine or Canine Immunomodulating Composition and Treatment Method”

### BACKGROUND OF THE INVENTION

1. Field of the Invention

The present invention relates to equine and canine immunomodulating compositions, equine and canine microbial infection treatment methods and equine and canine digestive tract regimens.

2. Background Information

β-Glucans occur naturally in the bran of grasses (Gramineae) such as barley, oats, rye and wheat, generally in amounts of about 7%, 5%, 2% and less than 1% respectively. They are recognized as a powerful natural immune stimulant. Additionally, beta-glucan moderates the release of sugars from the digestive system, helping to prevent the sugar highs and lows associated with intermittent feeding schedules of domesticated animals such as horses. For the performance horse such as a racehorse or show jumping horse, this may translate into greater focus and ability to concentrate during training and other tasks, as well as better overall health and well-being due to an enhanced immune system. Some difficulties with treating horses with β-Glucans is having effective amounts of β-Glucans in a format that can be easily and effectively provided to the horse. Similar problems arise with the supply of β-Glucans to canines.

Glucan extracted from yeast cell walls is also known to be a potent stimulator of the immune system. Studies have indicated that parenteral administration of glucan significantly improves host resistance to a wide variety of infectious disease caused by bacterial, fungal, viral, and parasitic organisms (DeLuzio, Trends in Pharmacological Science, 4:344-347, 1983). Yeast-derived beta-glucans have even been shown to provide resistance against anthrax (“Anthrax-protective effects of yeast beta 1,3 glucans,” Kournikakis B. et al., Med Gen Med. 2003 Mar. 21; 5 (1): 1). Glucan has also been shown to have potent antitumor activity (DeLuzio et al., Advances and Experimental Medicine and Biology, 21A:269-290, 1979). The mechanism by which glucan exerts its beneficial effects is believed to be by interaction with specific glucan receptors located on the macrophage cells. (Czop, Pathology & Immunopathology Res., 5:286-296, 1986).

The general method for the production of glucan from yeast involves extraction with alkali followed by extraction with acid (Hassid et all, Journal of the American Chemical Society, 63:295-298, 1941). Improved methods for isolating a purified water insoluble beta (1,3) glucan extract are disclosed in U.S. Pat. No. 5,223,491 which is incorporated herein by reference.

Naturally occurring glucans and those obtained by conventional prior art processes contain very high quantities of protein, chitin, and glycogen impurities. These glucans have historically been used in animal feeds and dietary supplements. The presence of hydrophobic contaminants such as proteins and glycogen can hinder the functional properties of the glucan matrix, rendering it less effective. In addition, the purity of the preparation should be an essential factor when considering the application of glucans in both the pharmaceutical and food industries.

U.S. Pat. No. 4,992,540, which is incorporated herein by reference, offers a technique of processing yeast cells to derive high purity glucans wherein the glucan product retains the three dimensional morphology of the intact yeast cell wall and has high water holding capacity. In turn, the glucan product may be further processed to yield glucans having improved or novel functional properties.

It is the object of the present invention to address the deficiencies of the prior art equine and canine immune support treatment options and provide a highly effective, easy to use immunomodulating treatment composition and method through the use of purified beta-glucans in novel dry feed supplements.

### SUMMARY OF THE INVENTION

One non-limiting aspect of the present invention provides a method of treating equine or canine microbial infections following the diagnosis of a microbial infection comprising the step of treating the equine or canine for a period at least long enough to prevent immediate recurrence of the microbial infection with effective amounts of an immunomodulating composition comprising a pre-packaged dry feed supplement comprising (a) an effective amount of a beta-glucan mixture comprising beta(1-3) glucan and beta(1-6) glucan, wherein said beta-glucan mixture is at least 60 percent purified.

The method of treating equine or canine microbial infections following the diagnosis of a microbial infection may further provide that the pre-packaged dry feed supplement further includes (b) an effective amount of at least one flavoring, such as apple; and (c) an effective amount of a sweetener, such as dextrose. The method of treating equine or canine microbial infections following the diagnosis of a microbial infection may provide that the treatment with the immunomodulating composition is for at least ten days. The method of treating equine or canine microbial infections following the diagnosis of a microbial infection may provide that the immunomodulating composition is used in combination with an effective amount of an antibiotic. The method of treating equine or canine microbial infections following the diagnosis of a microbial infection may provide that the immunomodulating composition further comprises an appetite stimulant. The method of treating equine microbial infections following the diagnosis of a microbial infection may provide a single-unit dosage of the immunomodulating composition comprises 500 to 2000 mg of a beta-glucan mixture comprising beta(1-3) glucan and beta(1-6) glucan. The immunomodulating composition may alternatively be used in combination with an effective amount of an antibiotic.

One non-limiting aspect of the present invention provides an equine or canine immunomodulating composition comprising a pre-packaged dry feed supplement comprising (a) an effective amount of a beta-glucan mixture comprising beta(1-3) glucan and beta(1-6) glucan, wherein said beta-glucan mixture is at least 60 percent purified, wherein the beta-glucan/composition weight ratio is in an amount of about 0.5 to about 2.0 beta-glucan to 7 composition, or even about 1.0 to about 1.5 beta-glucan to 7 composition, or about 1.2 beta-glucan to 7 composition.

The pre-packaged equine or canine immunomodulating composition may provide that the composition further comprises an appetite stimulant, wherein the appetite stimulant comprises an amino acid such as folic acid. The pre-packaged equine or canine immunomodulating composition may provide that a single-unit dosage of the composition contains the beta-glucan mixture in an amount of about 2 mg/kg. The pre-packaged equine or canine immunomodulating composition may further include (b) an effective amount of an antioxidant different from (a). The pre-packaged equine or canine immunomodulating composition may provide a single-unit daily dosage of the composition is about 7 grams.

One non-limiting aspect of the present invention provides an equine or canine immunomodulating composition comprising a pre-packaged supplement comprising: (a) an effective amount of a beta-glucan mixture comprising beta(1-3) glucan and beta(1-6) glucan, wherein said beta-glucan mixture is at least 60 percent purified; (b) an effective amount of an antioxidant different from (a); and (c) an effective amount of an emulsifier.

These and other advantages of the present invention will be clarified in the description of the preferred embodiments.

### DESCRIPTION OF THE PREFERRED EMBODIMENTS

It is noted that, as used in this specification and the appended claims, the singular forms “a,” “an,” and “the” include plural referents unless expressly and unequivocally limited to one referent.

For the purposes of this specification, unless otherwise indicated, all numbers expressing quantities of ingredients, reaction conditions, and other parameters used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

All numerical ranges herein include all numerical values and ranges of all numerical values within the recited numerical ranges. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The various embodiments and examples of the present invention as presented herein are understood to be illustrative of the present invention and not restrictive thereof and are non-limiting with respect to the scope of the invention.

In accordance with the present invention, an equine or canine immunomodulating composition comprising a pre-packaged dry feed supplement comprising an effective amount of a beta-glucan mixture comprising beta(1-3) glucan and beta(1-6) glucan, wherein said beta-glucan mixture is at least 60 percent purified, wherein the beta-glucan/composition weight ratio is in an amount of about 0.5 to about 2.0 beta-glucan to 7 composition. The ratio may be about 1.0 to about 1.5 beta-glucan to 7 composition, or about 1.2 beta-glucan to 7 composition.

The immunomodulating composition of the present invention comprises an effective amount of a beta-glucan mixture comprising beta(1-3)-glucan and beta(1-6)-glucan. Beta-glucans generally are known to provide enhanced immunity to infectious diseases by non-specifically activating a hosts' immune defense system. Activation is believed to occur by interaction of the beta-glucans with specific beta-glucan receptors on monocytes, thereby inducing the release of interleukin-1 (IL-1) and other cytokines and cellular mediators. Czop, (1986) Pathology and Immunipathology Research, 5:286-296; Williams et al., 1988, International Journal of Immunipharmacology, 9:261-267.

The beta-glucans suitable for use in the compositions of the invention which exhibit improved immunomodulation typically have a higher purity compared to existing, reported, naturally-occurring glucans. Glycogen, chitin and protein are undesirable contaminants found in naturally occurring and also in many conventionally obtained glucans. Such contaminants are believed to adversely affect the biological and hydrodynamic properties of the whole glucan particles, within the meaning and purpose of this invention.

Modified glucans useful in the present method include those prepared as described by Jamas et al. in U.S. Pat. Nos. 4,810,646, 5,028,703, 4,992,540, 5,037,972, and 5,082,936; the teachings of all of which are incorporated herein by reference, and in S. Jamas et al., Biotechnology and Bioengineering, 28:769-784 (1986). The beta-glucan mixture used in the composition of the present invention is at least 60 percent purified, using the processes described in the above-cited patents.

Modified beta-glucans from any strain of yeast can be used; however S. cerevisiae is the preferred strain. Modified beta-glucan may be produced, for example, from other strains of yeast, including Saccharomyces delbrueckii, Saccharomyces rosei, Saccharomyces microellipsodes, Saccharomyces carlsbergensis, Schizosaccharomyces pombe, Kluyveromyces lactis, Kluyveromyces fragilis, Kluyveromyces polysporus, Canadia albicans, Candida cloacae, Candida tropicalis, Candida utilis, Hansenula wingei, Hansenula arni, Hansenula henricii and Hansenula americana.

Modified beta-glucans which are particularly useful in the present composition are the highly branched beta-glucans derived from a mutant strain of yeast, Saccharomyces cerevisiae R4, (NRRL Y-15903), described by Jamas, et al., Biotechnology and Bioengineering, 28:769-784 (1986), and in U.S. Pat. No. 5,028,703. These modified glucans have enhanced in vitro and in vivo macrophage-activating properties when compared to naturally-occurring and conventional, commercial glucan preparations. More specifically, macrophage activating properties are related to the degree and type of beta(1-6) branching present on the glucan molecule. The modified glucans derived from the mutant yeast strain, S. cerevisiae R4, for example, have significantly more beta(1-6) branching than wild-type beta-glucans and demonstrate a stronger, more pronounced immune response than wild-type and non-modified glucan preparations.

The terms “naturally occurring glucans” and “wild-type glucans” are meant to include glucans and glucan preparations in which the glucan polymer itself or the organism which produces it (e.g., bacteria, yeast cells) has not been treated or modified to change the structure or concentration of the glucan. Such glucans contain high levels of protein, chitin, and glycogen impurities. Naturally occurring and wild-type glucans include previously reported commercial preparations such as, e.g., Zymosan, Lentinan and Glucan-P.

One non-limiting embodiment of the present invention an equine or canine immunomodulating composition is provided, comprising a pre-packaged, feed supplement comprising: (a) an effective amount of a beta-glucan mixture comprising beta(1-3) glucan and beta(1-6) glucan, wherein said beta-glucan mixture is at least 60 percent purified; (b) an effective amount of an antioxidant different from (a); and (c) an effective amount of an emulsifier.

The antioxidant of (b) used in the composition of the present invention may be any antioxidant known to be useful as additives in animal feeds. Typically the antioxidant comprises tocopherol and/or omega-3 fatty acids. One example of an omega-3 fatty acid is flaxseed oil. Studies in humans have found many health benefits with supplementation of omega-3 fatty acids in the diet. While research benefits for horses have not been as numerous, omega-3 supplementation shows potential to provide promising results.

Each molecule of fat or oil is derived from three fatty acid molecules and one glycerol molecule. A horse needs a certain amount of fat in its diet, and all fats contain omega-3 and omega-6 fatty acids. Horses require these two types of polyunsaturated fatty acids; examples of the most common acids are linoleic acid (an omega-6 fatty acid) and alpha-linolenic acid (an omega-3 fatty acid). These fats are termed essential fatty acids (EFA) because the horse's metabolism cannot synthesize them; they must be consumed in the diet to be provided. Although the exact EFA requirements for horses have not been established, they have been demonstrated as necessary for all animals and humans for many normal body functions. Deficiency of EFA in humans and animals includes hair loss, skin problems, and impaired immune function. Pasture grasses and hay, although containing only 2% to 3% fat, have greater concentrations of omega-3 than omega-6 fatty acids. Cereal grains, such as corn and oats, contain much higher levels of omega-6 than omega-3 fatty acids in reference to their total fat content. Both rice bran and soybean oils are higher in omega-3 and lower in omega-6 content than corn oil. And oils from sunflower, flax, and canola seeds contain the largest amount of omega-3's, with higher levels of omega-3 than omega-6 fatty acids. Flaxseed oil is the most concentrated plant source of omega-3 fatty acids or alpha-linoleic acid, also known as ALA. Fish oil is the greatest source of omega-3 fatty acids, with the highest ratio of omega-3:omega-6, and contains the omega-3's eicosapentaenoic acid (EPA) and docosahexanoic acid (DHA). Horses can convert ALA into DHA and EPA in their body tissues, which are used in various physiological functions, so these are not required.

The emulsifier of (c) used in the composition of the present invention may be any known food grade emulsifier, used in an effective amount, at least sufficient to prevent separation of the other ingredients. Typically the emulsifier comprises guar gum and/or sunflower oil.

In certain non-limiting embodiments of the present invention, the composition further comprises an appetite stimulant. Such stimulants can serve to counteract the natural appetite suppressing effect of the beta-glucan. Any suitable known appetite stimulating compounds may be used; amino acids such as folic acid are typically used.

In certain non-limiting embodiments of the present invention, the composition further comprises effective amounts of flavoring such as apples, which may be an artificial apple flavoring or natural apple, and a sweetener, particularly when used as an equine dietary supplement, to make the composition more palatable. The sweetener may be a natural sweetener such as dextrose or molasses in preferred embodiments. Apple and molasses mixtures for equine diets are known. There is a wide variety of mixtures of these ingredients that have been proposed. In general, ¼ cup of molasses to two apples represents an acceptable guideline for suitable ratios. The effective amount of apple/molasses mixture in the composition according to the invention is generally 5 to 60% by weight of the feed supplement. For dry feed prepackaged embodiments of the present invention using dextrose and flavoring additives, the weight percentage of the dextrose and flavoring/dry feed composition as a whole will be about 5 to about 6.5 dextrose and flavoring to 7 composition, or (5-6.5/7) weight ratio.

Typically a single-unit dosage of the immunomodulating composition of the present invention contains the beta-glucan mixture in an amount of 2±1 mg/kg. In those embodiments wherein the composition is an equine immunomodulating composition, a typical single-unit daily dosage of the composition may comprise: (a) 500 to 2000 mg, or 1000 to 1500 mg, and possibly about 1200 1000 mg, of a beta-glucan mixture comprising beta(1-3) glucan and beta(1-6) glucan, wherein said beta-glucan mixture is at least 60 percent purified. One nonlimiting embodiment of the invention may further include (b) 200 to 2000 mg of an antioxidant different from (a); and (c) 5 to 20 ml of an emulsifier.

Relative proportions for a canine formulation may be calculated based on the weight of the animal, using as a standard the average 2 mg/kg dosage of beta-glucan mixture.

The composition can include other vitamins, supplements or medicaments as known in the art. The preferred components and effective amounts are noted in the dry feed composition above. Ingredients may also be used to add additional flavoring and texture to the composition that is more acceptable or palatable to the animal, provided the daily dosage of the effective ingredients is maintained and the additives do not detrimentally affect such effectiveness as noted above.

The composition according to the present invention may be packaged in a dispensing container (e.g. 3-Gallon graduated bottle with drain-back pour spout) containing a preset number of unit dosages. For flowable compositions, or liquefied versions, this may be in a single or multiple dosage syringe. The packaging may be a large container filled with separable unit filled containers (e.g. foil pouches), whereby the user can simply empty the entire pouch without measuring unit dosages. The above composition is described in unit dosages amounts. There are many possible arrangements for the packaging of the composition according to the present invention; it is simply an important aspect of the present invention that the composition be pre-packaged for the end user.

The term “pre-packaged” within this application and in reference to the composition of the present invention references that the composition is preassembled in relative effective amounts. The user may be required to measure out one dosage, but need not combine the constituents. As discussed below, the pre-packaging may be in unit dosages such that measurement by the user is not required.

The term “dry feed” within this application in reference to the composition of the invention is understood as is conventionally known in the art and will reference a powder, granular, cubed or even pellet mixture of ingredients with no moisture content other than what may be from ambient conditions.

A “pill form” within the meaning of this invention is a subset of “dry feed” as it will be a dry feed composition formed into a pressed tablet (such as by adding a binder such as gelatin or the like), or a dry feed composition added within a dissolvable capsule, commonly made of gelatin. The pill form can be particularly useful with canine applications where the effective amounts of the ingredients of the present invention as scaled to the typical canine weights make the pill form an acceptable alternative. A daily pill form dosage of the present invention may be a selected multiple of pills, e.g. 3 pills daily, where the pills are generally associated with a given weight range of animal.

The phrase “effective amount” within this application and in reference to the composition refers to a desired unit dosage amount of the associated ingredient. The effective amounts per unit dosage of each ingredient are defined herein if not readily understood by those in the equine or canine care fields.

It is anticipated that the pre-packaged composition will contain multiple dosages, whereby the user will need to measure out, e.g. scoop out, a unit dosage. The effective amounts can be varied particularly if the treatment regimen is changed. For example, the effective amounts per unit dosage can be decreased if the daily treatments are increased.

The composition could alternatively be pre-packaged in single dosage containers which eliminate the need for the user to measure, but that is largely a packaging and shipping issue which is based upon whether the single use containers, or sub-containers, are prohibitively expensive. It is expected that the container could also include graduations thereon to assist in the measurement, as known in the dispensing container art, however a single serving size measuring scoop is often the most practical for bulk dry feed compositions.

According to certain embodiments of the present invention, a method of treating equine or canine microbial infections following the diagnosis of a microbial infection comprises the step of treating the equine or canine for a period at least long enough to prevent immediate recurrence of the microbial infection with effective amounts of an immunomodulating composition comprising a pre-packaged, dry feed supplement comprising (a) an effective amount of a beta-glucan mixture comprising beta(1-3) glucan and beta(1-6) glucan, wherein said beta-glucan mixture is at least 60 percent purified.

Often the treatment is for at least ten days, but may be of a longer or shorter duration as deemed necessary. Additionally, the composition of the present invention may be used as a daily dietary supplement to enhance the natural immune system.

In certain embodiments of the method of the present invention, the immunomodulating composition may be used in combination with an effective amount of an antibiotic as prescribed by a veterinarian. Suitable antibiotics may be any of those known and are chosen and administered based on the nature of the infection to be treated.

The phrase “diagnosis of a microbial infection” is not intended to be any specific diagnostic procedure, nor intended to suggest involvement of veterinarian personnel (although a veterinarian may be involved in the diagnosis and prescription of the composition of the present invention as therapy.) It is simply intended to suggest that the animal is suspected of already having an infection, which is opposed to where a therapy regimen is used to minimize the likelihood of microbial infection, e.g. prevention, where the animal currently likely does not have an infection. Note that the composition of the present invention may also be used as a regular dietary supplement or in a therapy regimen to boost the natural immune system.

The term “immediate recurrence” within this application and in reference to a microbial infection refers to a recurrence of a given infection within one month of completing a treatment regimen using the method of the present invention.

The dry feed composition of the present invention forms an all-natural product that promotes healthy equine and canine digestive function and enhances the immune system. The dry feed supplement will typically be supplied to the animal as a top dress of an effective amount (e.g., about 7 grams) on feed.

The composition of the present invention can be administered as a preventative treatment, for example, up to 72 hours before surgery or other event which will put the animal at risk for infection. The composition may also or alternatively be administered after the event, for example, for a period of up to 96 hours. By this treatment, the animal's non-specific and specific host defenses are stimulated by the composition to enhance the immune response. The treatment is also beneficial in fighting secondary infections.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. An equine or canine immunomodulating composition comprising a pre-packaged dry feed supplement comprising (a) an effective amount of a beta-glucan mixture comprising beta(1-3) glucan and beta(1-6) glucan, wherein said beta-glucan mixture is at least 60 percent purified, wherein the beta-glucan/composition weight ratio is in an amount of about 0.5 to about 2.0 beta-glucan to 7 composition.

2. The pre-packaged equine or canine immunomodulating composition of claim 1 further including (b) an effective amount of at least one flavoring; and (c) an effective amount of a sweetener.

3. The pre-packaged equine or canine immunomodulating composition of claim 1 wherein the composition further comprises an appetite stimulant, wherein the appetite stimulant comprises an amino acid.

4. The pre-packaged equine or canine immunomodulating composition of claim 3 wherein the amino acid comprises folic acid.

5. The pre-packaged equine or canine immunomodulating composition of claim 1 wherein a single-unit dosage of the composition contains the beta-glucan mixture in an amount of about 2 mg/kg.

6. The pre-packaged equine or canine immunomodulating composition of claim 1 further including (b) an effective amount of at least one flavoring, wherein the flavoring of (b) comprises apples.

7. The pre-packaged equine or canine immunomodulating composition of claim 6 further including (c) an effective amount of a sweetener, wherein the natural sweetener of (c) comprises dextrose.

8. The pre-packaged equine or canine immunomodulating composition of claim 1 further including (b) an effective amount of an antioxidant different from (a).

9. The pre-packaged equine or canine immunomodulating composition of claim 1 wherein the beta-glucan/composition weight ratio is in an amount of about 1 to about 1.5 beta-glucan to 7 composition.

10. The pre-packaged equine or canine immunomodulating composition of claim 9 wherein a single-unit daily dosage of the composition is about 7 grams.

11. The pre-packaged equine or canine immunomodulating composition of claim 9 wherein a single-unit dosage of the composition comprises 500 to 2000 mg of a beta-glucan mixture comprising beta(1-3) glucan and beta(1-6) glucan.

12. A method of treating equine or canine microbial infections following the diagnosis of a microbial infection comprising the step of treating the equine or canine for a period at least long enough to prevent immediate recurrence of the microbial infection with effective amounts of an immunomodulating composition comprising a pre-packaged dry feed supplement comprising (a) an effective amount of a beta-glucan mixture comprising beta(1-3) glucan and beta(1-6) glucan, wherein said beta-glucan mixture is at least 60 percent purified.

13. The method of treating equine or canine microbial infections following the diagnosis of a microbial infection of claim 12 wherein the pre-packaged dry feed supplement further includes (b) an effective amount of at least one flavoring; and (c) an effective amount of a sweetener.

14. The method of treating equine or canine microbial infections following the diagnosis of a microbial infection of claim 12 wherein the treatment with the immunomodulating composition is for at least ten days.

15. The method of treating equine or canine microbial infections following the diagnosis of a microbial infection of claim 12 wherein the immunomodulating composition is used in combination with an effective amount of an antibiotic.

16. The method of treating equine or canine microbial infections following the diagnosis of a microbial infection of claim 12 wherein the immunomodulating composition further comprises an appetite stimulant.

17. The method of treating equine or canine microbial infections following the diagnosis of a microbial infection of claim 12 wherein the pre-packaged dry feed supplement further includes (b) an effective amount of at least one flavoring, wherein the flavoring of (b) comprises apples, and pre-packaged dry feed supplement further includes (c) an effective amount of a sweetener, wherein the natural sweetener of (c) comprises dextrose.

18. The method of treating equine or canine microbial infections following the diagnosis of a microbial infection of claim 12 wherein the beta-glucans of the composition are obtained from the S. cerevisiae strain of yeast.

19. The method of treating equine or canine microbial infections following the diagnosis of a microbial infection of claim 12 wherein the method is a method of treating equine microbial infections and a single-unit dosage of the immunomodulating composition comprises 500 to 2000 mg of a beta-glucan mixture comprising beta(1-3) glucan and beta(1-6) glucan.

20. An equine or canine immunomodulating composition comprising a pre-packaged supplement comprising: (a) an effective amount of a beta-glucan mixture comprising beta(1-3) glucan and beta(1-6) glucan, wherein said beta-glucan mixture is at least 60 percent purified; (b) an effective amount of an antioxidant different from (a); and (c) an effective amount of an emulsifier
